(19) United States
(12) Patent Application Publication
(10) Pub. No.: US 2023/0332214 A1
(43) Pub. Date: Oct. 19, 2023

(54) METHOD OF MAPPING SPATIAL DISTRIBUTIONS OF CELLULAR COMPONENTS

(71) Applicant: CALIFORNIA INSTITUTE OF TECHNOLOGY, Pasadena, CA (US)

(72) Inventors: Long CAI, Pasadena, CA (US); Carsten H. TISCHBIREK, Pasadena, CA (US)

(21) Appl. No.: 18/074,369

(22) Filed: Dec. 2, 2022

Related U.S. Application Data
(60) Provisional application No. 63/285,836, filed on Dec. 3, 2021.

(51) Int. Cl. C12Q 1/6841; G01N 33/58

(52) U.S. Cl. CPC C12Q 1/6841; G01N 33/58

(57) ABSTRACT

This disclosure herein sets forth embodiments for a method for mapping the spatial distribution of one or more cellular components or cellular interactions by using barcodes to identify a cell or cellular component in the one or more cells, localizing, detecting, and mapping the spatial distribution of the one or more cellular components. The disclosure herein sets forth method to allow the mapping of spatial distributions of cellular components both intracellularly or intercellularly.

### CROSS-REFERENCE TO RELATED APPLICATION

The present application claims the benefit of U.S. Provisional Application No. 63/285,836, filed Dec. 3, 2021, and incorporates the entirety of that application by reference.

### STATEMENT REGARDING FEDERALLY SPONSORED RESEARCH OR DEVELOPMENT

This invention was made with government support under Grant No. MH116508 awarded by the National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 6, 2023, is named “439915.00110.xml” and is 11,169 bytes in size. The sequence listing contained in this .XML file is part of the specification and is hereby incorporated by reference herein in its entirety.

### FIELD OF THE INVENTION

The present disclosure generally relates to the field of mapping the spatial distribution of intracellular or intercellular components using barcode molecules. In particular, the embodiments disclosed herein relate to the design and characteristics of such barcode molecules, the creation of barcode diversity through defined molecular changes, barcode localization of cellular components, detection of cellular components, and mapping the cellular components in intracellular or intercellular interactions.

### BACKGROUND

A fundamental problem throughout multiple fields of biology is measuring how cellular components interact with each other intracellularly and intercellularly.

On the macroscale, cellular component distributions are essential for the organization of large numbers of cells for the formation of complex tissues during an organism's development, as well as diverse tissue functions such as immune responses or neural circuit formation. On the scale of individual cells, cellular component distributions contribute to the regulation of molecular signaling pathways, which regulate central aspects of cellular function such as metabolism or activity state. Cells interact with each other in numerous ways to exchange cellular components, either by close contact with neighboring cells or through secretion with distant cells, and such interactions can be either stable or transient. As cellular component distributions are essential for many cell and tissue functions, pathological changes to these interactions can lead to developmental defects as well as immunological and neurological diseases.

Existing methods to record cellular component distributions cab have severe limitations. For example, current approaches to record transport of cell components between cells or direct physical interactions between cells can be based on synthetic fluorescent dyes or fluorescent proteins that label an entire cell or a molecule inside the cell, molecules that induce the expression of fluorescent proteins after transfer between cells, reconstitution of a fluorescent molecule between two cells, or chemical tagging methods to record ligand-receptor interactions.

The existing methods can be limited to recording the interaction between defined pairs of cellular components. In some cases, they cannot establish interactions occurring between large groups of cellular components with single-cell resolution. Although the connectivity map of a complete nervous system was worked out in C. elegans, and more recently for the brain of Drosophila melanogaster, the existing methods to produce such maps can be resource and labor intensive, especially for larger tissues. Further, relating the mapped information to molecular characteristics of the cells within the neuronal network can be difficult. This is because the molecular characteristics of the cells often cannot be extracted from ultrastructural electron microscopy information.

New tools to map the intracellular and intercellular distributions of cellular components are needed. New tools that allow for the systematic recording of transient and stable interactions and the distributions of the cellular components between multiple neighboring and non-neighboring cells with single-cell specificity are needed. Further, new tools to record, in the context of a community of cells, the single cells of origin of intercellularly transferred molecules or organelles of interest, with minimal perturbations to cells and organisms, and ideally within the cells themselves are needed.

### SUMMARY OF THE INVENTION

The present disclosure provides methods for mapping the spatial distribution of one or more cellular components or cellular interactions by using barcodes to identify a cell or cellular component in the one or more cells, thereby localizing, detecting, and mapping the spatial distribution of the one or more cellular components or cellular interactions.

In some embodiments, there is provided a method for mapping spatial distribution of one or more cellular components comprising providing one or more cells. In certain embodiments, the method comprises providing one or more barcodes, wherein each barcode identifies a cell or cellular component in or near the one or more cells. In certain embodiments, the method comprises localizing each barcode to a site of a cellular component. In certain embodiments, the method comprises detecting each barcode and its location in the one or more cells. In certain embodiments, the method comprises recording one or more locations of each barcode to map the spatial distribution of the one or more cellular components.

In some embodiments, the methods comprise mapping a spatial distribution of cellular components or cellular interactions. In certain embodiments the methods comprise providing one or more cells. In certain embodiments, the methods comprise providing one or more barcodes, wherein each barcode identifies a cell or cellular component in or near the one or more cells. In certain embodiments, the methods comprise localizing each barcode to a site of a cellular component. In certain embodiments, the methods comprise detecting each barcode and its location in the one or more cells. In certain embodiments, the methods comprise recording one or more locations of each barcode to map the spatial distribution of the one or more cellular components.

In some embodiments, the methods comprise mapping of spatial relationships of one or more cells and cellular components. In certain embodiments, the methods comprise providing one or more cells. In certain embodiments, the methods comprise providing one or more barcodes, wherein each barcode identifies a cell or cellular component in or near the one or more cells. In certain embodiments, the methods comprise localizing each barcode to a site of a cellular component. In certain embodiments, the methods comprise detecting each barcode and its location in the one or more cells. In certain embodiments, the methods comprise recording one or more locations of each barcode to map the spatial distribution of the one or more cellular components.

In some embodiments, any of the preceding methods are useful for analyzing cell interactions in connective tissue, epithelial tissue, muscular tissue, nervous tissue, blood tissue, bone tissue, and lymph tissue. In certain embodiments, any of the preceding methods are useful to map neurological changes in an organism. In some embodiments, any of the preceding methods are useful for analyzing the changes in distributions of cellular components within a cell.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts an exemplary processes of mapping the spatial distributions of cellular components. In A, an barcode and a barcode changing molecule is introduced in to the cell. In B, a barcode changing molecule makes changes to the barcode. In C, the barcode is expressed in the cell. In D, the barcode is transported to a target region, which may include a cell surface receptor, synapse, organelle, or part of a secreted cellular component. In E, the spatial distribution of the barcodes are mapped using fluorophores and sequential imaging.

FIG. 2 depicts exemplary processes to edit barcodes. (A) depicts an exemplary embodiment of molecular changes to the barcode by an editing molecule that switches barcode recognition elements to an undetectable state. (B) depicts an editing molecule changing the barcode from an initially undetectable to a detectable state. (C) depicts an exemplary embodiment of molecular changes to the barcode molecule by an integrase such as Bxbl. (D) depicts an exemplary embodiment of molecular changes to the barcode molecule by a Cas9 nuclease guided by gRNA molecules. (E) depicts an exemplary embodiment of molecular changes to the barcode molecule by a Cas9 nuclease guided by gRNA molecules.

FIG. 3 depicts exemplary processes to design and detect barcodes. (A) illustrates example designs of barcode molecules, which are attached to localization molecules and detectable as proteins in cells. (B) illustrates detection of an example protein barcode with five recognition elements using five DNA oligo-conjugated antibodies. (C) illustrates example design of barcode molecules that are detectable as mRNA molecules in the cells. (D) illustrates detection of an example mRNA barcode with five recognition elements using five FISH probes.

FIG. 4 depicts an exemplary process of mapping the transport of a cellular component between cells. (A) depicts exemplary embodiments, illustrating barcode molecules attached to cell surface or cytoplasmic components of the cell, that can be used to measure transport through cell-cell contacts or cell junctions to neighboring cells. (B) depicts exemplary embodiments, illustrating barcode molecules attached to cell surface or cytoplasmic components of the cell, that can be used to measure transport through transient cell-cell contacts or cell junctions (1) to neighboring cells also when the interaction is no longer present (2). (C) depicts exemplary embodiments, illustrating barcodes attached to secretory cell components that can be used to measure secretion from cells to the extracellular space and uptake by other cells. (D) depicts exemplary embodiments, illustrating the barcodes localized to organelles in a cell that can be used to measure the transport of organelles to other cells in a cell population. (E) depicts exemplary embodiments, illustrating two distinct barcodes localized at the pre- and postsynaptic compartment of two neurons, that can be used to map neuronal connectivity.

FIG. 5 depicts an exemplary process of detecting barcodes in neuronal cells. (A) depicts exemplary embodiments, illustrating the detection of distinct barcodes using seqFISH imaging methods. (B) illustrates a group of neurons in intact tissue labeled with unique barcodes. (C) illustrates the morphology of neurons N3 and N4 of panel (b) reconstructed with ultrastructural details using electron microscopy methods (left panel). (D) shows an exemplary synaptic connection between neurons N1 (purple) and N2 (green). Overlapping points corresponding to synaptic connections between two neurons are shown in red.

FIG. 6 depicts an exemplary process of using transgenic elements to label the synapses of neurons in C. elegans. (A) illustrates the transgenic elements used to label synapses in a C. elegans worm with a barcode molecule. (B) illustrates the anatomy of the worm head with locations of neuronal processes labeled as dendritic regions. (C) shows FISH imaging data of the same region as shown in (B) of a transgenic worm expressing both elements shown in (A) mRNA barcode was detected using fluorescent FISH probes (magenta) and cell nuclei are visualized by DAPI staining (cyan).

FIG. 7 provides an exemplary process of using transgenic barcode elements to label the synapses and cell bodies of neurons in the fruit fly Drosophila melanogaster, and illustrates protein barcode detection in cell bodies and synaptic terminals of the same cells. (A) illustrates an exemplary embodiment where transgenic elements were used to label the synapses of a subset of neurons in the fly brain. A GH146 promoter was used to control expression of a barcode (abbreviated as BC) molecule linked to a synaptic protein DSyd-1 together with a scaffold molecule, in this exemplary embodiment a GFP (green fluorescent protein) molecule. To generate the image, a whole brain was dissected from an adult Drosophila melanogaster, fixed with PFA, and labeled with antibodies. Scale bar=50 um. (B) illustrates the anatomical location of the exemplary labeled projection neurons as a reference for the brain shown in (A). (C-J) illustrate magnified images of the anatomical locations labeled as white rectangles in (A). Scale bars=10 um. (C) illustrates the anatomical location of the magnified region of the mushroom body calyx for reference. (D) shows a confocal microscopy image of the DSyd-1 protein barcode labeled with a fluorescent antibody against an epitope of the protein barcode. (E) shows a confocal microscopy image of endogenous presynaptic proteins labeled with an anti-Brp antibody. (F) illustrates the location of cell nuclei with a DAPI staining, corresponding to Kenyon cells surrounding the calyx. The signals of the protein barcode and endogenous synaptic protein overlap. (G) illustrates the anatomical location of the magnified region of the antennal lobe for reference. (H) shows a confocal microscopy image of the DSyd-1 protein barcode labeled with a fluorescent antibody against an epitope of the protein barcode. (I) shows a confocal microscopy image of endogenous presynaptic proteins labeled with an anti-Brp antibody. (J) illustrates the location of cell nuclei with a DAPI staining, corresponding to antennal lobe projection neurons.

FIG. 8 depicts an exemplary process of mapping cellular components in Drosophila. (A) shows an agarose gel image of PCR-amplified barcode sequences that were extracted from flies expressing a barcode molecule and a Bxbl integrase. (8) illustrates imaging of edited protein barcodes in transgenic flies by using two antibodies against different recognition sites of the barcode. DAPI staining is included (second to right panel) to show locations of cell nuclei in the overlay of all channels (rightmost panel).

FIG. 9 depicts an exemplary process of mapping cellular components in neuronal cells. (A) illustrates anatomical structures of the drosophila mushroom body calyx, in which presynaptic boutons of antennal lobe projection neurons connect to postsynaptic Kenyon cells, whose cell bodies surround the calyx. (B) illustrates how a protein barcode is expressed and transported together with a postsynaptic protein in Kenyon cells in adult drosophila brains. The postsynaptic protein barcode is labeled with an antibody specific to one of its epitope tags. (C) illustrates the locations of presynaptically expressed protein barcodes labeled with an antibody specific for the presynaptic protein barcode. Scale bar in (A-C)=10 um. (D) illustrates the locations of cell nuclei in the same region labeled with the fluorescent dye DAPI. (E) illustrates imaging data of a magnified synapse in the Mushroom body calyx as indicated by the white rectangle shown in (B) and (C). The same location in the calyx is shown for postsynaptic barcode protein (left panel) and presynaptic barcode protein (right panel). The pre- and postsynaptically expressed protein barcodes are localized in a characteristic pattern for synapses of projection neurons and Kenyon cells. Scale bar=1 um. (F) illustrates a schematic of mushroom body synapse structures and the expected locations for postsynaptic barcode molecules in relation to presynaptic boutons.

FIG. 10, provides an exemplary process of using antibodies labeled with oligonucleotides to sequentially image individual barcode elements at the same synaptic region in a PFA-fixed Drosophila melanogaster brain. (A) illustrates an exemplary embodiment where a GH146 promoter was used to control expression of a barcode molecule, which is linked to a synaptic protein DSyd-1 together with a scaffold GFP molecule. The image shows the presynaptic region of the GH146-positive antennal lobe projection neurons found in the mushroom body calyx. (B) illustrates the sequential imaging individual barcode elements using antibody conjugated to unique oligonucleotide sequences. During the experiment, antibody-conjugated oligonucleotide sequences are bound by specific, fluorophore-labeled oligonucleotides sequences (‘readout oligos’). After removal of the fluorescent signal recorded in hybridization round 1, the next readout oligo is bound to another epitope tag, until all epitope tags are imaged. Exemplary images correspond to the white rectangle shown in (A) and show the signal for all 6 unique epitope tags imaged with 6 unique readout oligos at the same synaptic location. Scale bar=1 urn.

FIG. 11 provides an exemplary process of labeling organelles with barcode molecule in a transgenic model organism. (A) illustrates a mitochondria-targeting peptide attached to a GFP molecule. (B) illustrates an exemplary image of a larval Drosophila melanogaster muscle labeled with a mitochondrially targeted GFP molecule. Scale bar=10 um. (C) illustrates a magnification corresponding to the white rectangle in (B), showing fluorescently labeled mitochondria in the tissue. (D) illustrates a mitochondria-targeting peptide attached to a barcode molecule that has, in this exemplary embodiment, 6 unique epitope tags. (E) illustrates an exemplary image of a larval Drosophila melanogaster muscle labeled with a mitochondrially targeted protein barcode molecule, similar to the mitochondrially targeted GFP shown in (B-C). (F) illustrates a magnification corresponding to the white rectangle in (E), showing fluorescently labeled mitochondria in the tissue.

FIG. 12 provides an exemplary process of mapping the diversity of spatially distributed neuronal barcodes. (A) illustrates an exemplary timeline of an imaging experiment with transgenic Drosophila melanogaster, which express barcode molecules that are transported to synapses and a heat shock-dependent recombinase to create barcode diversity. In this example, flies are heat shocked during their larval and pupal stages. At the adult stage, fly brains are dissected, PFA-fixed, labeled with antibodies and imaged using fluorescence microscopy. (B) illustrates exemplary confocal microscopy image of a GFP molecule used as a scaffold protein for the protein barcode in the presynaptic region of the L3 neurons in the optic lobe of the fly. (C-F) illustrate exemplary confocal microscopy images of protein-barcode signals in the same presynaptic region of the L3 neurons in the optic lobe of the fly as shown in (B). (G) illustrates an exemplary process of barcode information extracted from the epitope tag imaging rounds shown in (C-F) for ROIs (regions of interest) 1-3 indicated in panels (C-F) with “0” corresponding to no signal detected at the ROI and “1” corresponding to signal detected at the ROI.

### DETAILED DESCRIPTION

The following description is presented to enable one of ordinary skill to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges.

Oligonucleotides can be of various lengths. In particular embodiments, oligonucleotides can range from about 2 to about 200 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to for a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to one or more specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As disclosed herein, the term “barcode” generally refers to a symbol sequence of a labels produced by methods described herein. The barcode sequence typically is of a sufficient length and uniqueness to identify a molecular target in a single cell. In certain embodiments, the barcode lengths are between 100 bp to 20,000 bp.

As disclosed herein, the term “cellular components” refers to a target selected from transcripts, RNA, DNA loci, chromosomes, DNA, protein, antibodies, lipids, glycans, cellular targets, organelles, synapses, cell-to-cell junctions, cellular component boundaries and any combinations thereof.

As disclosed herein, the term “distribution” refers to the location of a cellular component within a cell. In certain embodiments, the term “distributions” also refers to the interactions between cellular components and a cell, between other cellular components.

As disclosed herein, the term “epitope” refers to the part of protein to which an antibody or protein recognizing the part of the protein may bind.

As disclosed herein, the term “mapping” refers to detecting a barcode linked to a cellular component and identifying its position intracellularly or extracellularly.

As disclosed herein, the term “binding” refers to the interaction of two molecules. In certain embodiments, “binding” may refer to the hybridization of two nucleotide sequences. In certain embodiments, “binding” may refer to a protein-nucleotide interaction. In certain embodiments, “binding” may refer to a protein-protein interaction.

### EMBODIMENTS

Certain embodiments are useful to map the spatial distribution of one or more cellular components by providing one or more barcodes to identify a cell or cellular component in the one or more cells to record the locations of the barcode to map the spatial distribution of the one or more cellular components.

Certain embodiments are useful to map the spatial distribution of cellular components or cellular interactions by providing one or more barcodes to identify a cell or cellular component in the one or more cells to record the locations of the barcode to map the spatial distribution of the one or more cellular components.

Certain embodiments are useful to map the of spatial relationships of one or more cells and cellular components by providing one or more barcodes to identify a cell or cellular component in the one or more cells to record the locations of the barcode to map the spatial distribution of the one or more cellular components.

Certain embodiments are useful to map the distribution of a cellular component that is intracellularly distributed. In some embodiments, the method comprises mapping the distribution of a cellular component that is distributed intercellularly. In certain embodiments, the method comprises mapping a cellular component that is distributed both intracellularly and intercellularly. In some embodiments, the method comprises mapping the distribution of at least one cellular component that is distributed intracellularly. In some embodiments, the method comprises mapping the distribution of at least one cellular component that is distributed intercellularly.

Certain embodiments are useful to map the history or past events of the cellular components that are intracellularly or intercellularly distributed. In some embodiments, the method comprises mapping a cellular component and relating it to the history of a cell or a group of cells. Certain embodiments are useful to map the history of intracellularly and intercellularly distributed cellular components in individual cells or a group of cell in relation to the history of intracellularly and intercellularly distributed cellular components of other cells.

### BARCODES

The methods disclosed comprise mapping the distribution of the cellular components or cellular interactions of cells with barcodes. The barcodes can be any barcodes deemed useful by the person of skill. In certain embodiments, the barcodes comprise oligonucleotides, peptides, proteins, carbohydrates, or combinations thereof. The barcodes disclosed comprise unique barcodes, each linked to a distinct cellular component, that are capable of being detected and identified. Each barcode provides a unique identification barcode of a cellular component that is detectable among the other barcodes created by mapping techniques.

In some embodiments, the barcodes comprise unique combinations of barcode elements. In certain embodiments, the unique combinations comprise combinatorially arranged barcode elements.

In some embodiments, each barcode comprises one or more barcode elements. In some embodiments, the barcode comprises two or more barcode elements. In some embodiments, the barcode comprises three or more barcode elements. In some embodiments, the barcode comprises four or more barcode elements. In some embodiments, the barcode comprises five or more barcode elements. In some embodiments, the barcode comprises six or more barcode elements. In some embodiments, the barcode comprises seven or more barcode elements. In some embodiments, the barcode comprises eight or more barcode elements. In some embodiments, the barcode comprises nine or more barcode elements. In some embodiments, the barcode comprises ten or more barcode elements. In some embodiments, the barcode comprises fifteen or more barcode elements. In some embodiments, the barcode comprises twenty or more barcode elements. In some embodiments, the barcode comprises one or more barcode elements.

In certain embodiments, each barcode element in a barcode is separated from each other by spacer nucleotides. In some embodiments, the barcode elements in a barcode are separated by amino acid spacers.

In certain embodiments, each barcode element comprises one or more error correction elements. In certain embodiments, each error correction element comprises a repeat of a barcode element in a combination of barcode elements. In some embodiments, the error correction elements are according to a Hamming code (Hamming, R, 1950.)

In some embodiments, the barcode elements comprise barcode recognition sequences that are recognized by one or more recognition molecules. In some embodiments, the barcode recognition sequences are adjacent to or are flanked by one or more barcode elements capable of recruiting a molecule capable of changing the barcode elements.

In some embodiments, the barcode recognition sequence comprises a nucleotide sequence, a protein coding nucleotide sequence, multiple repeats of the same protein coding nucleotide sequence, a codon optimized protein coding nucleotide sequence, a protein or any combination thereof.

In some embodiments, the barcode recognition sequences of any of the previous embodiments, are linked to the N-terminal, C-terminal, or N- and C-terminals of a cellular component.

In some embodiments, the barcode elements are detected by proteins, peptides, antibodies, oligonucleotides, or any combination thereof, binding each element. In certain embodiments, the barcode elements are detected by an oligonucleotide labeled with a fluorophore capable of binding to complementary barcode element nucleotide sequence. In certain embodiments, the barcode elements are detected by an antibody labeled with a fluorophore. In certain embodiments, the antibody is an anti-FLAG, anti-Myc, or anti-Human influenza hemagglutinin (HA), anti-V5, anti-GFP (green fluorescent protein), anti-GST (glutathione-S-transferase), anti-β-GAL (β-galactosidase), anti-S1 (spike protein s1), anti-HSV1gD (Herpes simplex virus-1 gD), anti-AUS, anti-VSV-G (vesicular stomatitis virus G), anti-E, anti-S, anti-Spot, anti-Nano, anti-PA, anti-E2, anti-T7, anti-GCN4, anti-Glu-Glu, anti-m B-Tag, anti-RAP, anti-AU1, anti-ALFA, anti-V5, anti-OLLAS, anti-HSV, anti-Inntag6, anti-Inntag10, anti-Protein C (autoprothrombin IIA), anti-KT3, anti-TK15, anti-Softag1, anti-NE, anti-HAT (Histidine aminotransferase 1), or anti-Rho (rhodopsin). In some embodiments, the barcode elements are composed of TALENs (transcription activator-like effector nucleases) or ZFNs (Zinc finger nucleases). In certain embodiments, the TALENs or ZFNs are detected with a DNA sequence specific for the TALENs or ZFNs. In some embodiments, this TALEN- or ZFN-specific DNA sequence is attached to a single-stranded oligonucleotide sequence that is detected by an oligonucleotide labeled with a fluorophore.

In some embodiments, the cellular components are mapped using techniques comprising sequential hybridization, FISH, or seqFISH. In certain embodiments, barcodes comprise sequential hybridization barcodes, FISH barcodes, or seqFISH barcodes as disclosed in International PCT Patent Application No. PCT/US2014/036258, file Apr. 30, 2014, and titled MULTIPLEX LABELING OF MOLECULES BY SEQUENTIAL HYBRIDIZATION BARCODING, the entire contents of which are herein incorporated by reference in its entirety for all purposes.

### NUCLEOTIDE BARCODES

In some embodiments, one or more barcode elements in a barcode comprises a nucleotide sequence. In some embodiments, each element in the one or more barcode elements is different from each other barcode element. In some embodiments, each barcode element in a barcode comprises a nucleotide sequence that is the same as each other barcode element in a barcode. In some embodiments, each barcode element in a barcode comprises a nucleotide sequence that is the same or different from each other barcode element.

In some embodiments, the barcode comprises a nucleotide sequence having a length between about 100 bp to 20,000 bp. In some embodiments, the barcode comprises a nucleotide sequence having a length between about 200 bp to 20,000 bp. In some embodiments, the barcode comprises a nucleotide sequence having a length between about 300 bp to 20,000 bp. In some embodiments, the barcode comprises a nucleotide sequence having a length between about 400 bp to 20,000 bp. In some embodiments, the barcode comprises a nucleotide sequence having a length between about 500 bp to 20,000 bp. In some embodiments, the barcode comprises a nucleotide sequence having a length between about 600 bp to 20,000 bp. In some embodiments, the barcode comprises a nucleotide sequence having a length between about 700 bp to 20,000 bp. In some embodiments, the barcode comprises a nucleotide sequence having a length between about 800 bp to 20,000 bp. In some embodiments, the barcode comprises a nucleotide sequence having a length between about 900 bp to 20,000 bp. In some embodiments, the barcode comprises a nucleotide sequence having a length between about 1,000 bp to 20,000 bp. In some embodiments, the barcode comprises a nucleotide sequence having a length between about 1,500 bp to 20,000 bp. In some embodiments, the barcode comprises a nucleotide sequence having a length between about 2,000 bp to 20,000 bp. In some embodiments, the barcode comprises a nucleotide sequence having a length between about 2,500 bp to 20,000 bp. In some embodiments, the barcode comprises a nucleotide sequence having a length between about 3,000 bp to 20,000 bp. In some embodiments, the barcode comprises a nucleotide sequence having a length between about 3,500 bp to 20,000 bp. In some embodiments, the barcode comprises a nucleotide sequence having a length between about 4,000 bp to 20,000 bp. In some embodiments, the barcode comprises a nucleotide sequence having a length between about 4,500 bp to 20,000 bp. In some embodiments, the barcode comprises a nucleotide sequence having a length between about 5,000 bp to 20,000 bp. In some embodiments, the barcode comprises a nucleotide sequence having a length between about 5,500 bp to 20,000 bp. In some embodiments, the barcode comprises a nucleotide sequence having a length between about 6,000 bp to 20,000 bp. In some embodiments, the barcode comprises a nucleotide sequence having a length between about 6,500 bp to 20,000 bp. In some embodiments, the barcode comprises a nucleotide sequence having a length between about 7,000 bp to 20,000 bp. In some embodiments, the barcode comprises a nucleotide sequence having a length between about 7,500 bp to 20,000 bp. In some embodiments, the barcode comprises a nucleotide sequence having a length between about 8,000 bp to 20,000 bp. In some embodiments, the barcode comprises a nucleotide sequence having a length between about 8,500 bp to 20,000 bp. In some embodiments, the barcode comprises a nucleotide sequence having a length between about 9,000 bp to 20,000 bp. In some embodiments, the barcode comprises a nucleotide sequence having a length between about 9,500 bp to 20,000 bp. In some embodiments, the barcode comprises a nucleotide sequence having a length between about 10,000 bp to 20,000 bp. In some embodiments, the barcode comprises a nucleotide sequence having a length between about 11,000 bp to 20,000 bp. In some embodiments, the barcode comprises a nucleotide sequence having a length between about 12,000 bp to 20,000 bp. In some embodiments, the barcode comprises a nucleotide sequence having a length between about 13,000 bp to 20,000 bp. In some embodiments, the barcode comprises a nucleotide sequence having a length between about 14,000 bp to 20,000 bp. In some embodiments, the barcode comprises a nucleotide sequence having a length between about 15,000 bp to 20,000 bp. In some embodiments, the barcode comprises a nucleotide sequence having a length between about 16,000 bp to 20,000 bp. In some embodiments, the barcode comprises a nucleotide sequence having a length between about 17,000 bp to 20,000 bp. In some embodiments, the barcode comprises a nucleotide sequence having a length between about 18,000 bp to 20,000 bp. In some embodiments, the barcode comprises a nucleotide sequence having a length between about 19,000 bp to 20,000 bp.

In some embodiments, a barcode recognition sequence comprises a nucleotide sequence having a length between about 10 bp to 200 bp. In some embodiments, a barcode recognition sequence comprises a nucleotide sequence having a length between about 10 bp to 180 bp. In some embodiments, a barcode recognition sequence comprises a nucleotide sequence having a length between about 10 bp to 160 bp. In some embodiments, a barcode recognition sequence comprises a nucleotide sequence having a length between about 10 bp to 140 bp. In some embodiments, a barcode recognition sequence comprises a nucleotide sequence having a length between about 10 bp to 160 bp. In some embodiments, a barcode recognition sequence comprises a nucleotide sequence having a length between about 10 bp to 140 bp. In some embodiments, a barcode recognition sequence comprises a nucleotide sequence having a length between about 10 bp to 140 bp. In some embodiments, a barcode recognition sequence comprises a nucleotide sequence having a length between about 10 bp to 100 bp. In some embodiments, a barcode recognition sequence comprises a nucleotide sequence having a length between about 10 bp to 80 bp. In some embodiments, a barcode recognition sequence comprises a nucleotide sequence having a length between about 10 bp to 60 bp. In some embodiments, a barcode recognition sequence comprises a nucleotide sequence having a length between about 10 bp to 40 bp. In some embodiments, a barcode recognition sequence comprises a nucleotide sequence having a length between about 10 bp to 20 bp. In some embodiments, a barcode recognition sequence comprises a nucleotide sequence having a length between about 20 bp to 200 bp. In some embodiments, a barcode recognition sequence comprises a nucleotide sequence having a length between about 20 bp to 180 bp. In some embodiments, a barcode recognition sequence comprises a nucleotide sequence having a length between about 20 bp to 160 bp. In some embodiments, a barcode recognition sequence comprises a nucleotide sequence having a length between about 20 bp to 140 bp. In some embodiments, a barcode recognition sequence comprises a nucleotide sequence having a length between about 20 bp to 160 bp. In some embodiments, a barcode recognition sequence comprises a nucleotide sequence having a length between about 20 bp to 120 bp. In some embodiments, a barcode recognition sequence comprises a nucleotide sequence having a length between about 20 bp to 120 bp. In some embodiments, a barcode recognition sequence comprises a nucleotide sequence having a length between about 20 bp to 100 bp. In some embodiments, a barcode recognition sequence comprises a nucleotide sequence having a length between about 20 bp to 80 bp. In some embodiments, a barcode recognition sequence comprises a nucleotide sequence having a length between about 20 bp to 60 bp. In some embodiments, a barcode recognition sequence comprises a nucleotide sequence having a length between about 20 bp to 40 bp.

In some embodiments, a barcode recognition sequence comprises a nucleotide sequence having a length between about 10 bp to 1000 bp. In some embodiments, a barcode recognition sequence comprises a nucleotide sequence having a length between about 20 bp to 100 bp. In some embodiments, a barcode recognition sequence comprises a nucleotide sequence having a length between about 40 bp to 100 bp. In some embodiments, a barcode recognition sequence comprises a nucleotide sequence having a length between about 60 bp to 1000 bp. In some embodiments, a barcode recognition sequence comprises a nucleotide sequence having a length between about 80 bp to 1000 bp. In some embodiments, a barcode recognition sequence comprises a nucleotide sequence having a length between about 100 bp to 1000 bp. In some embodiments, a barcode recognition sequence comprises a nucleotide sequence having a length between about 120 bp to 1000 bp. In some embodiments, a barcode recognition sequence comprises a nucleotide sequence having a length between about 140 bp to 1000 bp. In some embodiments, a barcode recognition sequence comprises a nucleotide sequence having a length between about 160 bp to 1000 bp. In some embodiments, a barcode recognition sequence comprises a nucleotide sequence having a length between about 180 bp to 1000 bp. In some embodiments, a barcode recognition sequence comprises a nucleotide sequence having a length between about 200 bp to 1000 bp. In some embodiments, a barcode recognition sequence comprises a nucleotide sequence having a length between about 220 bp to 1000 bp. In some embodiments, a barcode recognition sequence comprises a nucleotide sequence having a length between about 240 bp to 1000 bp. In some embodiments, a barcode recognition sequence comprises a nucleotide sequence having a length between about 260 bp to 1000 bp. In some embodiments, a barcode recognition sequence comprises a nucleotide sequence having a length between about 280 bp to 1000 bp. In some embodiments, a barcode recognition sequence comprises a nucleotide sequence having a length between about 300 bp to 1000 bp. In some embodiments, a barcode recognition sequence comprises a nucleotide sequence having a length between about 320 bp to 1000 bp. In some embodiments, a barcode recognition sequence comprises a nucleotide sequence having a length between about 340 bp to 1000 bp. In some embodiments, a barcode recognition sequence comprises a nucleotide sequence having a length between about 360 bp to 1000 bp. In some embodiments, a barcode recognition sequence comprises a nucleotide sequence having a length between about 380 bp to 1000 bp. In some embodiments, a barcode recognition sequence comprises a nucleotide sequence having a length between about 400 bp to 1000 bp. In some embodiments, a barcode recognition sequence comprises a nucleotide sequence having a length between about 420 bp to 1000 bp. In some embodiments, a barcode recognition sequence comprises a nucleotide sequence having a length between about 440 bp to 1000 bp. In some embodiments, a barcode recognition sequence comprises a nucleotide sequence having a length between about 460 bp to 1000 bp. In some embodiments, a barcode recognition sequence comprises a nucleotide sequence having a length between about 480 bp to 1000 bp. In some embodiments, a barcode recognition sequence comprises a nucleotide sequence having a length between about 500 bp to 1000 bp. In some embodiments, a barcode recognition sequence comprises a nucleotide sequence having a length between about 520 bp to 1000 bp. In some embodiments, a barcode recognition sequence comprises a nucleotide sequence having a length between about 540 bp to 1000 bp. In some embodiments, a barcode recognition sequence comprises a nucleotide sequence having a length between about 560 bp to 1000 bp. In some embodiments, a barcode recognition sequence comprises a nucleotide sequence having a length between about 580 bp to 1000 bp. In some embodiments, a barcode recognition sequence comprises a nucleotide sequence having a length between about 600 bp to 1000 bp. In some embodiments, a barcode recognition sequence comprises a nucleotide sequence having a length between about 620 bp to 1000 bp. In some embodiments, a barcode recognition sequence comprises a nucleotide sequence having a length between about 640 bp to 1000 bp. In some embodiments, a barcode recognition sequence comprises a nucleotide sequence having a length between about 660 bp to 1000 bp. In some embodiments, a barcode recognition sequence comprises a nucleotide sequence having a length between about 680 bp to 1000 bp. In some embodiments, a barcode recognition sequence comprises a nucleotide sequence having a length between about 700 bp to 1000 bp. In some embodiments, a barcode recognition sequence comprises a nucleotide sequence having a length between about 720 bp to 1000 bp. In some embodiments, a barcode recognition sequence comprises a nucleotide sequence having a length between about 740 bp to 1000 bp. In some embodiments, a barcode recognition sequence comprises a nucleotide sequence having a length between about 760 bp to 1000 bp. In some embodiments, a barcode recognition sequence comprises a nucleotide sequence having a length between about 780 bp to 1000 bp. In some embodiments, a barcode recognition sequence comprises a nucleotide sequence having a length between about 800 bp to 1000 bp. In some embodiments, a barcode recognition sequence comprises a nucleotide sequence having a length between about 820 bp to 1000 bp. In some embodiments, a barcode recognition sequence comprises a nucleotide sequence having a length between about 840 bp to 1000 bp. In some embodiments, a barcode recognition sequence comprises a nucleotide sequence having a length between about 860 bp to 1000 bp. In some embodiments, a barcode recognition sequence comprises a nucleotide sequence having a length between about 880 bp to 1000 bp. In some embodiments, a barcode recognition sequence comprises a nucleotide sequence having a length between about 900 bp to 1000 bp. In some embodiments, a barcode recognition sequence comprises a nucleotide sequence having a length between about 920 bp to 1000 bp. In some embodiments, a barcode recognition sequence comprises a nucleotide sequence having a length between about 940 bp to 1000 bp. In some embodiments, a barcode recognition sequence comprises a nucleotide sequence having a length between about 960 bp to 1000 bp. In some embodiments, a barcode recognition sequence comprises a nucleotide sequence having a length between about 980 bp to 1000 bp.

In some embodiments, a barcode recognition sequence comprises multiple repeats of the nucleotide sequences of any of the previous embodiments.

In some embodiments, each barcode element is capable of binding a labeled oligonucleotide. In certain embodiments, each labeled oligonucleotide is capable of binding to each barcode element of the barcode. In certain embodiments, each label on the labeled oligonucleotide comprises a fluorophore.

### PROTEIN BARCODES

In some embodiments, one or more barcode elements in a barcode comprise a protein sequence. In some embodiments, each barcode element in a barcode is different from each other barcode element. In some embodiments, each barcode element in a barcode comprises a protein sequence that is the same as each other barcode element in a barcode. In some embodiments, each barcode element in a barcode comprises a protein sequence that is the same or different from each other barcode element.

In some embodiments, one or more barcodes recognition sequences comprise one or more amino acid sequences. In some embodiments, a barcode recognition sequence comprises two or more amino acid sequences. In some embodiments, a barcode recognition sequence comprises three or more amino acid sequences. In some embodiments, a barcode recognition sequence comprises four or more amino acid sequences. In some embodiments, a barcode recognition sequence comprises five or more amino acid sequences. In some embodiments, a barcode recognition sequence comprises six or more amino acid sequences. In some embodiments, a barcode recognition sequence comprises seven or more amino acid sequences. In some embodiments, a barcode recognition sequence comprises eight or more amino acid sequences. In some embodiments, a barcode recognition sequence comprises nine or more amino acid sequences. In some embodiments, a barcode recognition sequence comprises ten or more amino acid sequences. In some embodiments, a barcode recognition sequence comprises fifteen or more amino acid sequences. In some embodiments, a barcode recognition sequence comprises twenty or more amino acid sequences. In some embodiments, a barcode recognition sequence comprises thirty or more amino acid sequences.

In some embodiments, a barcode recognition sequence comprises one or more repeated amino acid sequences. In some embodiments, a barcode recognition sequence comprises two or more repeated amino acid sequences. In some embodiments, a barcode recognition sequence comprises three or more repeated amino acid sequences. In some embodiments, a barcode recognition sequence comprises four or more repeated amino acid sequences. In some embodiments, a barcode recognition sequence comprises five or more repeated amino acid sequences. In some embodiments, a barcode recognition sequence comprises six or more repeated amino acid sequences. In some embodiments, a barcode recognition sequence comprises seven or more repeated amino acid sequences. In some embodiments, a barcode recognition sequence comprises eight or more repeated amino acid sequences. In some embodiments, a barcode recognition sequence comprises nine or more repeated amino acid sequences. In some embodiments, a barcode recognition sequence comprises ten or more repeated amino acid sequences. In some embodiments, a barcode recognition sequence comprises fifteen or more repeated amino acid sequences. In some embodiments, a barcode recognition sequence comprises twenty or more repeated amino acid sequences. In some embodiments, a barcode recognition sequence comprises thirty or more repeated amino acid sequences.

In certain embodiments, the amino acid sequences of any of the previous embodiments, comprise epitope tags. In certain embodiments, the epitope tags comprise 6×His, FLAG, HA, Myc, V5, GFP, GST, β-GAL, Luciferase, MBP, RFP, and VSV-G, S1, HSV1gD, AU5, VSV-G, E, S, Spot, Nano, PA, E2, T7, GCN4, Glu-Glu,m B-Tag, RAP, AU1, ALFA, OLLAS, HSV, Inntag6, Inntag10, Protein C, KT3, TK15, Softag1, NE, HAT, Rho, or any combination thereof. In certain embodiments, the epitope tags comprise GluGlu (EE), ALFA, E, DYKDDDDK (SEQ ID NO: 1), VSVG, OLLAS, HA, E2, S, T7, V5, MYC, or any combination thereof. In certain embodiments, the barcode recognition sequence comprises repeated, variable nucleotide sequences, coding for the same amino acid sequence. In certain embodiments, the epitope tags are detected by antibodies of any of the previous embodiments directed against an epitope.

### Spacer Nucleotides and Amino Acids

In some embodiments, the barcode elements in a barcode are separated by spacer nucleotides. In some embodiments, the barcode elements in a barcode are separated by amino acid spacers.

In some embodiments, the barcodes elements in a barcode are separated by nucleotide spacers comprising a nucleotide sequence having a length between about 1 bp to 100 bp. In certain embodiments, the barcodes elements in a barcode are separated by nucleotide spacers comprising a nucleotide sequence having a length between about 5 bp to 100 bp. In some embodiments, the barcodes elements in a barcode are separated by nucleotide spacers comprising a nucleotide sequence having a length between about 10 bp to 100 bp. In some embodiments, the barcodes elements in a barcode are separated by nucleotide spacers comprising a nucleotide sequence having a length between about 15 bp to 100 bp. In some embodiments, the barcodes elements in a barcode are separated by nucleotide spacers comprising a nucleotide sequence having a length between about 20 bp to 100 bp. In some embodiments, the barcodes elements in a barcode are separated by nucleotide spacers comprising a nucleotide sequence having a length between about 25 bp to 100 bp. In some embodiments, the barcodes elements in a barcode are separated by nucleotide spacers comprising a nucleotide sequence having a length between about 30 bp to 100 bp. In some embodiments, the barcodes elements in a barcode are separated by nucleotide spacers comprising a nucleotide sequence having a length between about 40 bp to 100 bp. In some embodiments, the barcodes elements in a barcode are separated by nucleotide spacers comprising a nucleotide sequence having a length between about 50 bp to 100 bp. In some embodiments, the barcodes elements in a barcode are separated by nucleotide spacers comprising a nucleotide sequence having a length between about 60 bp to 100 bp. In some embodiments, the barcodes elements in a barcode are separated by nucleotide spacers comprising a nucleotide sequence having a length between about 70 bp to 100 bp. In some embodiments, the barcodes elements in a barcode are separated by nucleotide spacers comprising a nucleotide sequence having a length between about 80 bp to 100 bp. In some embodiments, the barcodes elements in a barcode are separated by nucleotide spacers comprising a nucleotide sequence having a length between about 90 bp to 100 bp.

In some embodiments, the barcode elements in a barcode are separated by amino acids spacers comprising an amino acid sequence having a length between about 1 and 20 amino acids. In some embodiments, the barcode elements in a barcode are separated by amino acids spacers comprising an amino acid sequence having a length between about 2 and 20 amino acids. In some embodiments, the barcode elements in a barcode are separated by amino acids spacers comprising an amino acid sequence having a length between about 3 and 20 amino acids. In some embodiments, the barcode elements in a barcode are separated by amino acids spacers comprising an amino acid sequence having a length between about 4 and 20 amino acids. In some embodiments, the barcode elements in a barcode are separated by amino acids spacers comprising an amino acid sequence having a length between about 5 and 20 amino acids. In some embodiments, the barcode elements in a barcode are separated by amino acids spacers comprising an amino acid sequence having a length between about 6 and 20 amino acids. In some embodiments, the barcode elements in a barcode are separated by amino acids spacers comprising an amino acid sequence having a length between about 7 and 20 amino acids. In some embodiments, the barcode elements in a barcode are separated by amino acids spacers comprising an amino acid sequence having a length between about 8 and 20 amino acids. In some embodiments, the barcode elements in a barcode are separated by amino acids spacers comprising an amino acid sequence having a length between about 9 and 20 amino acids. In some embodiments, the barcode elements in a barcode are separated by amino acids spacers comprising an amino acid sequence having a length between about 10 and 20 amino acids. In some embodiments, the barcode elements in a barcode are separated by amino acids spacers comprising an amino acid sequence having a length between about 15 and 20 amino acids.

### Editing a Barcode

In some embodiments, the barcodes can edited. In certain embodiments, the barcodes can be edited by any molecule deemed useful by the person of skill. In some embodiments, the method comprises a step of using a molecule to alter the barcode.

In certain embodiments, the molecule comprises an editing molecule. In certain embodiments, the recognition molecule comprises a nuclease, recombinase, integrase, Cas9 nuclease, or any combination thereof. In certain embodiments, the recognition molecule comprises an endonuclease, a zinc finger nuclease, a recombinase, a Cas9 enzyme, tag-specific guide RNA, integrase, small molecule or any combination thereof.

In some embodiments, the molecule alters one or more barcode elements by inserting nucleotides, deleting nucleotides, substituting one or more nucleotides, inserting amino acids, deleting amino acids, substituting or more amino acids. In certain embodiments, the editing protein alters a barcode element to render the barcode element non-functional. In certain embodiments, the editing protein alters a barcode element to render the barcode element functional. In certain embodiments, the editing protein alters the barcode to render an epitope on a barcode non-functional. In certain embodiments, the editing protein alters the barcode to render an epitope on a barcode functional.

In certain embodiments, an editing protein alters an epitope tag without inducing amino acid frameshifts affecting downstream translation of other barcodes and/or target proteins. In one particular embodiment, the editing protein alters an epitope tag by using an Bxb1 integrase to prevent translation of an epitope of a barcode by removing the epitope from the barcode by excision using the attB and attP recombination sites flanking the epitope.

In some embodiments, each barcoded cellular component of any of the previous embodiments, can be inherited through progeny cells. In certain embodiments, each edited barcoded cellular component of any of the previous embodiment can be inherited through progeny cells.

### Scaffold Proteins and Nucleic Acids

In some embodiments, the barcodes or barcode recognition sequences of any of the previous embodiments are linked to one or more scaffold proteins capable of localizing the barcodes deemed useful by the person of skill in the art. In some embodiments, the barcodes or barcode recognition sequences of any of the previous embodiments are linked to one or more nucleic acids capable of localizing the barcodes deemed useful by the person of skill in the art.

In some embodiments, the one or more barcodes of any of the previous embodiments are linked to one or more scaffold proteins capable of localizing the barcodes. In certain embodiments, the scaffold proteins comprise green fluorescent protein (GFP). In certain embodiments, the scaffold proteins comprise GFP variants. In certain embodiments, the GFP variants comprise genetically encoded Ca²⁺ indicators (GECIs). In any of the previous embodiments, the scaffolds aid in the expression of the barcodes.

In some embodiments, the scaffold proteins are capable of binding oligonucleotides, proteins, peptides, antibodies, small molecules, or any combination thereof for mapping cellular components.

In some embodiments, the one more barcodes are linked to nucleic acid scaffolds capable of localizing the barcodes. In some embodiments, the nucleic acid scaffold comprises an RNA scaffold. In some embodiments, the nucleic acid scaffold comprises an RNA loop capable of binding proteins. In certain embodiments, the RNA scaffold comprises a PP7 or an MS2 viral RNA sequence.

In some embodiments, the nucleic acid scaffolds are capable of binding oligonucleotides, proteins, peptides, antibodies, small molecules, or any combination thereof for mapping cellular components.

### Incorporating the Barcodes into Cells

In some embodiments, the barcodes can be produced exogenously or endogenously by any method deemed useful by the person of skill in the art. In some embodiments, the barcodes are introduced by into one or more cells by any method deemed useful by the person of skill in the art.

In some embodiments, the barcodes comprise an exogenous set synthesized outside the cell. In some embodiments, the barcode is an exogenous set synthesized outside the cell.

In certain embodiments, the barcodes prepared outside the one or more cells are introduced into one or more cells by transfection, transformation, transduction, conjugation techniques, or any combination thereof.

In some embodiments, the barcodes are expressed in the cell. In certain embodiments, the barcodes are expressed as RNA and translated to protein. In certain embodiments, the barcodes are expressed under control of constitutively active promoter sequences, cell-specific promoter sequences, drug-inducible, light-inducible, or temperature-dependent promoter-sequences, or any combination thereof.

In some embodiments, the barcodes are introduced to two or more cells, wherein the two or more cells interact with each other in a tissue sample. In some embodiments, the tissue sample comprises epithelial tissue, connective tissue, muscle tissue, nervous tissue, blood, bone, lymph, or any combination thereof. In some embodiments, the barcodes are introduced to a subject

In certain embodiments, the one or more barcodes are linked to endogenous or synthetic components localized to the surface of the cell. In certain embodiments, the one or more barcodes are linked to endogenous or synthetic secreted components of the cell. In certain embodiments, the one or more barcodes are linked to endogenous or synthetic components localized in one or more subcellular compartments of one or more cells.

In some embodiments, the barcodes comprise one or more orthogonal barcode sets. In certain embodiments, each barcode in the orthogonal barcode set comprises one or more detection sites that differ in binding from the one or more binding sites of a different barcode in the set. In some embodiments, the barcodes comprise one or more orthogonal barcode sets.

In one particular embodiment, the one or more barcodes are linked to endogenous or synthetic components localized to a synaptic sub-compartment of the cell.

### Detecting Barcodes

In some embodiments, the barcodes can be detected by any technique deemed suitable by a person of skill in the art. In certain embodiments, the detecting the one or more barcodes comprises using imaging, sequencing, mass spectrometry, or any combination thereof.

In some embodiments, the nucleotide barcodes are detected by binding oligonucleotides labeled with a fluorophore to each barcode element in a barcode. In some embodiments, the protein barcodes are detected by binding proteins labeled with a fluorophore to each barcode element in a barcode. In some embodiments, the protein barcodes are detected by binding antibodies labeled with a fluorophore to each barcode element in a barcode. In some embodiments, the protein barcodes are detected by binding proteins bound to oligonucleotides labeled with a fluorophore to each barcode element in a barcode. In some embodiments, the protein barcodes are detected by binding antibodies bound to oligonucleotides labeled with a fluorophore to each barcode element in a barcode. In some embodiments, the method comprises fluorescence detection.

In some embodiments, the barcodes of any of the preceding embodiments are detected by using microscopy techniques. In certain embodiments, the barcodes of any of the preceding embodiments are detected by using fluorescence microscopy.

### Mapping the Cellular Components

In some embodiments, the cellular component can be mapped by any technique deemed suitable by a person of skill in the art. In some embodiments, the method comprises mapping the distribution of subcellular compartments that travel intracellularly or intercellularly, or both.

In some embodiments, the method comprises mapping intercellular and/or intracellular spatial distribution of one or more cellular components. In certain embodiments, the method comprises mapping the intercellular and/or intracellular components spatially distributed between two or more cells in a population. In some embodiments, the intercellular spatial distribution of one or more cellular component are related by the localized barcodes. In some embodiments, the recording and mapping of intercellular and/or intracellular spatial distribution of one or cellular components between individual or a plurality of cells comprises localizing barcodes in close proximity to or within one or more cells. In certain embodiments, the recording and mapping of intercellular and/or intracellular spatial distribution of one or more cellular components of cells comprises relating the molecular properties of one or more transport molecules to one or more locations of the barcodes. In certain embodiments, the recording and mapping of intercellular and/or intracellular interactions comprises relating the location of one or more barcodes originating in different cells to each other. In certain embodiments, the recording and mapping of intercellular spatial distribution of one or more cellular components comprises quantifying the amount of one or more barcodes in close proximity to or within cells.

In some embodiments, the recording and mapping intercellular spatial distributions of one or more cellular components comprises applying a set of probes to one or more cells, wherein the probes bind the barcodes. In some embodiments, the probes comprise antibodies or peptides that can bind specific recognition sequences of the barcode. In some embodiments, the probes are selected from DNA, RNA, small molecules that can bind specific recognition sequences of the barcode, peptides, proteins, antibodies, and combinations thereof. In some embodiments, the method comprises one or more probes bind to one or more target sites on one or more barcodes. In some embodiments, each probe is associated with a label capable of a signal upon binding between the probe and its corresponding target site on one or more barcodes. In certain embodiments, the label is a fluorophore.

In some embodiments, the recording and mapping comprise characterizing molecular changes of the plurality of barcode elements based on the absence and presence of signals. In certain embodiments, a signal indicates an unaltered recognition site of a barcode and absence of a signal indicates an alteration at the recognition site of the barcode. In certain embodiments, a signal indicates an altered recognition site of a barcode and absence of a signal indicates an unalteration at the recognition site of the barcode. In certain embodiments, the alteration comprises mutations or chemical modifications to the barcodes that make the barcode either functional or non-functional. In certain embodiments, the alteration comprises a deletion, insertion, rearrangement, or any combination thereof of one or more nucleotides within the barcodes.

In some embodiments, the method comprises sequential hybridization to detect barcodes. In certain embodiments, the barcodes are mapped by fluorescence in situ hybridization (FISH). In certain embodiments, the barcodes are mapped by sequential fluorescence in situ hybridization techniques (seqFISH). Certain techniques for analyzing the biological samples are known. See, for example, International PCT Patent Application No. PCT/US2014/036258, file Apr. 30, 2014, and titled MULTIPLEX LABELING OF MOLECULES BY SEQUENTIAL HYBRIDIZATION BARCODING, the entire contents of which are herein incorporated by reference in its entirety for all purposes.

In some embodiments, the seqFISH method comprises performing a first contacting step that comprises contacting a sample, wherein the sample comprises one or more cells, or wherein the sample is processed from one or more cells, or wherein the sample comprises a plurality of nucleic acids with a first plurality of detectably labeled oligonucleotides, each of which targets a nucleic acid in the sample and is labeled with a detectable moiety, so that the composition comprises at least: (i) a first oligonucleotide targeting a first nucleic acid in the sample and labeled with a first detectable moiety; and (ii) a second oligonucleotide targeting a second nucleic acid in the sample and labeled with a second detectable moiety. In some embodiments, the method comprises imaging the cell sample after the first contacting step so that interaction by the first plurality of oligonucleotides with their targets is detected. In some embodiments, the method comprises repeating the contacting and imaging steps, each time with a plurality of detectably labeled oligonucleotides wherein at least one target nucleic acid contacted by multiple pluralities of detectably labeled oligonucleotides is targeted with a different detectable moiety labeling of oligonucleotides in at least one of the pluralities. In some embodiments, the method comprises optionally, performing additional rounds of contacting and imaging prior or in between or after steps (a)-(e) for error correction with block codes.

In some embodiments, the seqFISH method comprises performing a first contacting step that comprises contacting a cell sample, wherein the sample comprises one or more cells, or wherein the sample, comprises a plurality of target proteins or target nucleic acids, with a first plurality of detectably labeled proteins or oligonucleotides or combinations thereof, each of which targets a target protein or target nucleic acid and is labeled with a detectable moiety, so that the composition comprises at least: (i) a first protein or oligonucleotide or combination thereof, targeting a first target protein or target nucleic acid in the sample and labeled with a first detectable moiety; and (ii) a second protein or oligonucleotide or combination thereof, targeting a second target protein or target nucleic acid in the sample and labeled with a second detectable moiety. In some embodiments, the seqFISH method comprises (b) imaging the sample after the first contacting step so that interaction by proteins or oligonucleotides or combinations thereof, of the first plurality of detectably labeled proteins or oligonucleotides or combinations thereof, with their targets is detected. In some embodiments, the seqFISH method comprises repeating the contacting and imaging steps, each time with a plurality of detectably labeled proteins or oligonucleotides or combinations thereof, wherein at least one target protein or target nucleic acid in the sample is contacted by multiple pluralities of detectably labeled proteins or oligonucleotides or combinations thereof, is targeted with a different detectable moiety labeling of proteins or oligonucleotides or combinations thereof in at least one of the pluralities. In some embodiments, the method comprises optionally, performing additional rounds of contacting and imaging prior or in between or after steps (a)-(e) for error correction with block codes.

In some embodiments, the seqFISH method comprises performing a first contacting step that comprises contacting a sample, wherein the sample comprises one or more cells, or wherein the sample comprises a plurality of target proteins or target nucleic acids, with a first plurality of intermediate proteins or intermediate oligonucleotides or combinations thereof, each of which: (i) targets a target protein or target nucleic acid in the sample and is optionally labeled with a detectable moiety; and (ii) optionally, comprises an overhang sequence after hybridization with the target; so that the composition comprises at least: (i) a first intermediate protein or oligonucleotide or combination thereof, targeting a target first protein or target nucleic acid in the plurality of target proteins or target nucleic acids and optionally labeled with a first detectable moiety; and (ii) a second intermediate protein or oligonucleotide or combination thereof, targeting a second target protein or target nucleic acid in the plurality of target proteins or target nucleic acids and optionally labeled with a second detectable moiety. In some embodiments, the seqFISH method comprises contacting the first plurality of intermediate proteins or intermediate oligonucleotides with a first plurality of detectably labeled proteins or oligonucleotides or combinations thereof comprising at least: (i) a first detectably labeled protein or oligonucleotide or combination thereof, targeting a set of the intermediate proteins or oligonucleotides or combination thereof; and (ii) optionally, a second detectably labeled protein or oligonucleotide or combination thereof, targeting a set of the intermediate proteins or oligonucleotides or combination thereof. In some embodiments, the seqFISH method comprises imaging the sample after contacting the first plurality of intermediate proteins or intermediate oligonucleotides with one or more detectably labeled proteins or oligonucleotides or combinations thereof, so that the interaction of the intermediate protein or intermediate oligonucleotide with their targets is detected. In some embodiments, the seqFISH method comprise repeating the contacting and imaging steps, each time with a plurality of detectably labeled proteins or oligonucleotides or combinations thereof that target intermediate proteins or oligonucleotides or combinations thereof bound to target proteins or target nucleic acids, wherein at least one-intermediate protein or oligonucleotide or combination thereof is targeted with a different detectable moiety labeling of proteins or oligonucleotides or combinations thereof in at least one of the pluralities. In some embodiments, the seqFISH method comprises optionally, performing additional rounds of contacting the sample intermediate proteins or oligonucleotides or combinations thereof. In some embodiments, the seqFISH method comprises optionally, performing additional rounds of contacting and imaging prior or in between or after steps (a)-(e) for error correction with block codes.

In some embodiments, the seqFISH method comprises contacting the sample with a plurality of intermediate proteins or intermediate oligonucleotides or combinations thereof, each of which: (i) targets a target protein or target nucleic acid in the sample and is optionally labeled with a detectable moiety; and (ii) optionally, comprises an overhang sequence after hybridization with the target. In certain embodiments, the seqFISH method comprises optionally, imaging the cell so that interaction between the intermediate oligonucleotides with their targets is detected.

In some embodiments, the seqFISH method comprises an error correction round performed by selecting from block codes such as Hamming codes, Reed-Solomon codes, Golay codes, or any combination thereof.

In some embodiments, the seqFISH method comprises removing probes by using stripping reagents, wash buffers, photobleaching, chemical bleaching, and any combinations thereof.

### Fluorophores

In some embodiments, the oligonucleotides or proteins used to map the cellular components in any of the previous embodiments comprise a fluorophore. In some embodiments, the fluorophores used to perform mapping of cellular components can be any technique deemed suitable by a person of skill in the art.

In certain embodiments, the fluorophores include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red). In certain embodiments, the fluorophores include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium(II), Texas Red, TSQ, Umbelliferone, or Yellow fluorescent protein. In certain embodiments, the fluorophores include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750. In certain embodiments, the fluorophores comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the label comprises a nanomaterial. In some embodiments, the label is a nanoparticle. In some embodiments, the label is or comprises a quantum dot. In some embodiments, the fluorophore is a quantum dot. In some embodiments, the label comprises a quantum dot. In some embodiments, the label is or comprises a gold nanoparticle. In some embodiments, the label is a gold nanoparticle. In some embodiments, the label comprises a gold nanoparticle.

In some embodiments, the term “label” in any of the previous embodiments, may be synonymous with fluorophore.

In certain embodiments, the label in any of the previous embodiments comprises a barcode.

In some embodiments, the methods of any of the previous embodiments comprise one or more probes are linked with a plurality of labels that produce different signals.

### EXAMPLES

### Example 1

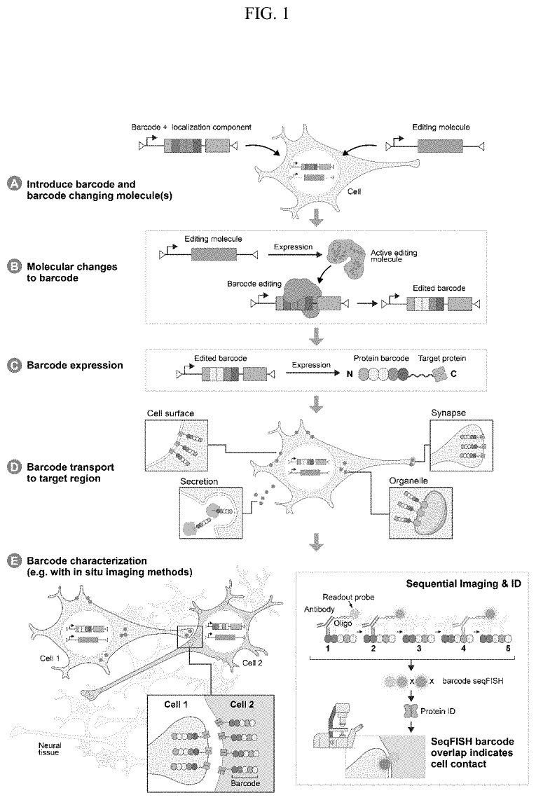

This example, as illustrated in FIG. 1, provides an exemplary process of mapping the spatial distributions of cellular components.

In 1, a barcode and a barcode changing molecule are introduced into a cell. The barcode is operably linked to a cellular component. The barcode and barcode changing molecule can be operably transcribed within the nucleus of the cell. Alternatively, the barcode and barcode changing molecule can be operably translated with the cytoplasm of the cell.

In 2, the barcode changing molecule makes changes to the barcode to produce an edited barcode.

In 3, the changed barcode is translated to produce a protein barcode linked to a cellular component, in this case a target protein.

In 4, the barcode linked to the cellular component is transported to a target region, which may include a cell surface receptor, synapse, organelle, or part of a secreted cellular component. The cellular component integrates into the cell as a cell surface receptor, synapse, organelle, or part of a secreted component.

In 5, the spatial distribution of the barcodes are mapped using fluorophores and sequential imaging. The mapping may utilize antibodies capable of binding oligonucleotides that bind a probe. The use of seqFISH and multiple rounds of hybridization uniquely maps each barcode linked to a cellular component accurately.

### Example 2

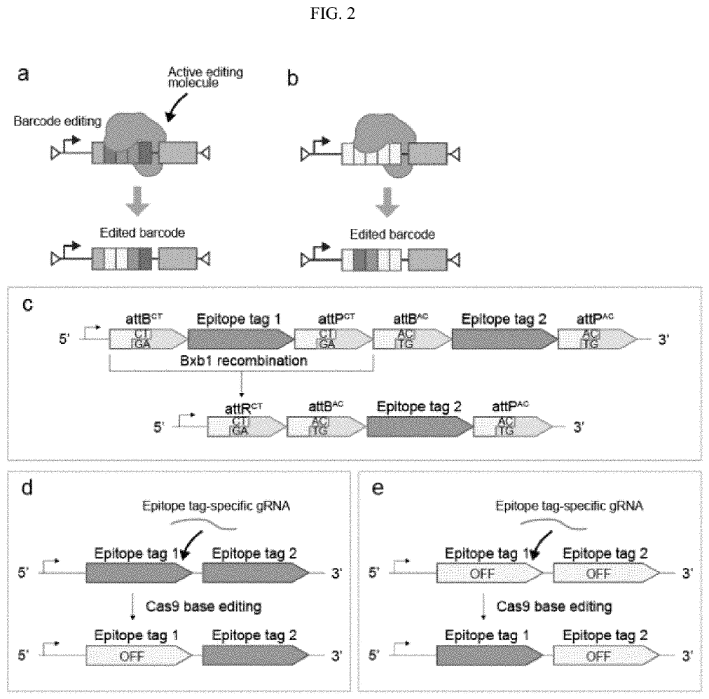

This example, as illustrated in FIG. 2, provides an exemplary processes to change a barcode by using an editing molecule.

(A) illustrates an exemplary embodiment of molecular changes to the barcode by an editing molecule that switches barcode recognition elements to an undetectable state. Combinations of detectable and undetectable elements create barcode diversity. (B) depicts an exemplary embodiment of molecular changes by an editing molecule to change the barcode from an initially undetectable to a detectable state.

(C) illustrates an exemplary embodiment of molecular changes to the barcode molecule by an integrase such as Bxbl. The integrase leads to recombination of specific attB and attP sites flanking a recognition element, in this example an epitope tag sequence, resulting in the deletion of the epitope tag sequence. In case the barcode is a protein, molecular changes to the recognition elements do not induce frameshifts interfering with more C-terminal amino acid sequences of the barcode.

(D) illustrates an exemplary embodiment of molecular changes to the barcode molecule by a Cas9 nuclease guided by gRNA molecules. The change impairs binding of a recognition molecule, for example by inducing one or multiple amino acid changes to inhibit epitope recognition by barcode detection antibodies.

(E) illustrates an exemplary embodiment of molecular changes to the barcode molecule by a Cas9 nuclease guided by gRNA molecules. The molecular change allows binding of a recognition molecule.

### Example 3

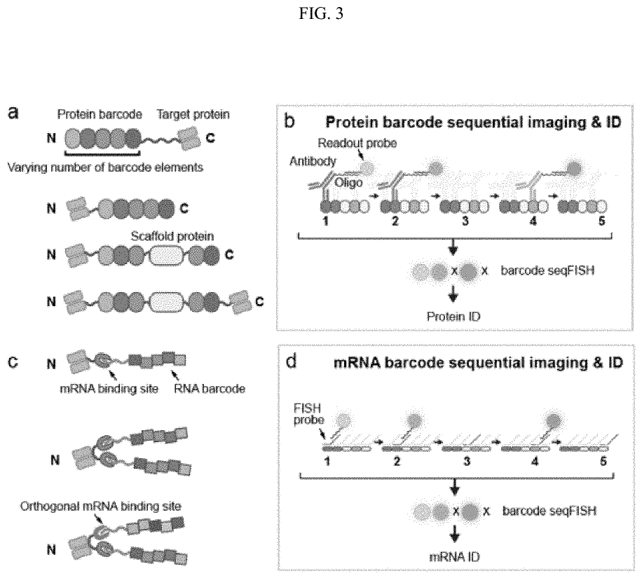

This example, as illustrated in FIG. 3, provides an exemplary designs of barcode molecules.

(A) illustrates an exemplary embodiment of barcode molecules designs, which are attached to localization molecules and detectable as proteins in cells. Barcode elements can vary in number based on the required barcode diversity. Barcode fusions with proteins can be N- or C-terminal or within the protein, and can also contain domains such as scaffolds for the barcode or additional localization, protein stability or degradation domains.

(B) illustrates an exemplary embodiment of a protein barcode with five recognition elements using five DNA oligo-conjugated antibodies. Sequences for elements 3′ and 5′ of the example barcode are molecularly changed so they cannot be detected with antibodies. FISH probes labeled with fluorescent dyes are used to detect the oligo-conjugated antibodies. FISH imaging data is used to determine the ID of the protein barcode.

(C) illustrates an exemplary embodiment of barcode molecules that are detectable as mRNA molecules in the cells. mRNA barcode molecules are transported via a mRNA binding protein attached to the localization protein. Multiple orthogonal mRNA binding proteins and recognition sequences for the mRNA-protein binding can be combined.

(D) illustrates an exemplary embodiment of a mRNA barcode with five recognition elements using five FISH probes. Hybridization probes are detected during sequential imaging rounds with fluorescently labeled FISH readout probes. FISH imaging data is used to determine the ID of the mRNA barcode.

### Example 4

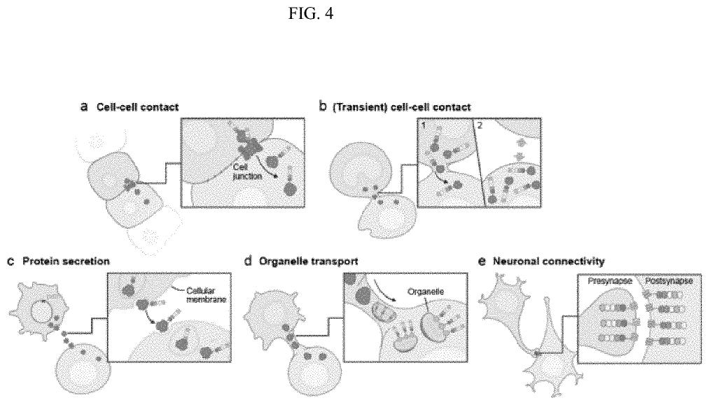

This example, as illustrated in FIG. 4, provides an exemplary designs of barcode molecules for the use of analysing cell contacts, protein secretion, organelle transport, and neuronal connectivity.

(A) illustrates s an exemplary embodiment, illustrating barcode molecules attached to cell surface or cytoplasmic components of the cell, that can be used to measure transport through cell-cell contacts or cell junctions to neighboring cells.

(B) illustrates exemplary embodiments, illustrating barcode molecules attached to cell surface or cytoplasmic components of the cell, that can be used to measure transport through transient cell-cell contacts or cell junctions (1) to neighboring cells also when the interaction is no longer present (2).

(C) illustrates exemplary embodiments, illustrating barcodes attached to secretory cell components that can be used to measure secretion from cells to the extracellular space and uptake by other cells.

(D) illustrates exemplary embodiments, illustrating the barcodes localized to organelles in a cell that can be used to measure the transport of organelles to other cells in a cell population.

(E) illustrates exemplary embodiments, illustrating two distinct barcodes localized at the pre- and postsynaptic compartment of two neurons, that can be used to map neuronal connectivity.

### Example 5

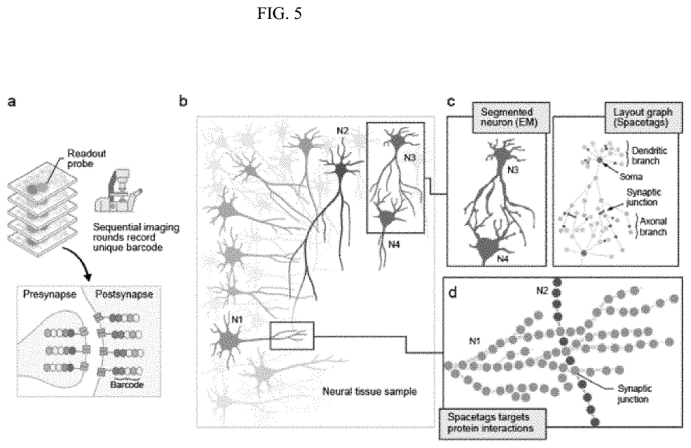

This example, as illustrated in FIG. 5, provides an exemplary process of detecting barcodes in neuronal cells.

(A) illustrates exemplary embodiments, illustrating the detection of distinct barcodes using imaging methods. SeqFISH with multiple imaging rounds is used to identify barcodes localized at pre- and post-synaptic sites. The data from seqFISH can be combined with data from other imagining methods such as electron microscopy.

(C) illustrates an exemplary embodiment where the morphology of neurons N3 and N4 of panel (b) are reconstructed with ultrastructural details using electron microscopy methods (left panel). In contrast, using protein barcodes (here called “spacetags”) to measure cell-cell interactions focuses on the synaptic connections between the neurons.

The data from various imagining methods can be combined with other imaging markers to label the cell body, nucleus, and molecular markers to specify neuron types.

### Example 6

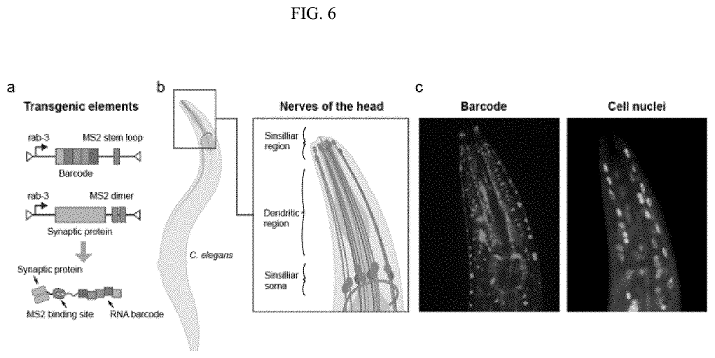

This example, as illustrated in FIG. 6, provides an exemplary process of using transgenic elements to label the synapses of neurons in C. elegans.

HCR-FISH experiments were conducted as previously described (Shah et al. 2016) with modifications made for whole-mount C. elegans worms and performed at room temperature unless specified otherwise

C. elegans N2 worms were injected with plasmids encoding for a dimer mRNA binding protein MS2 attached to the synaptic protein nlg-1, and a barcode with an MS2 hairpin under control of a neuronal rab-3 promoter. Transgenic worms were selected using a fluorescent microscope and washed in 1.7 ml tubes until visible contaminants were removed. Worms were fixed with 4% PFA and stored in 100% methanol overnight at −20° C. After rehydration, the worm cuticle was reduced with freshly prepared TCEP solution and oxidized in freshly made H₂O₂ solution. Worms were cleared in 8% SDS, and cleared worms were hybridized with a primary FISH probe against the target barcode mRNA. Primary FISH probes were designed with a binding site for HCR amplifiers. Following hybridization, worms were washed with 50% formamide and incubated with gel solution containing acrylamide/bis-acrylamide and VA-044 initiator. A circular spacer was attached to coverslips functionalized with bind-silane and Poly-D-lysine to avoid crushing the worms and to keep the gel solution in place. Worms in gel solution were transferred to the coverslips, and gently pressed down with a second coverslip. Gel polymerization was completed at 37° C. in a humidification chamber filled with nitrogen gas. The second coverslip, spacer and excess gel were removed from the sample, and a custom-designed flow cell was attached to the coverslip to cover the worms. Worms were digested with proteinase K overnight. HCR amplifier oligos conjugated to Alexa Fluor 647 were added to the samples according to previously published protocol (1), which were then washed to remove excess and non-specifically bound HCR amplifier oligos. Next, worms were stained with DAPI and covered with an anti-bleaching buffer for imaging. Imaging was performed with a microscope (Leica DMi8) equipped with a confocal scanner unit (Yokogawa CSU-W1), a sCMOS camera (Andor Zyla 4.2 Plus), and a 40× oil objective lens (Leica 1.40 NA).

(A) illustrates an exemplary embodiment where transgenic elements were used to label the synapses in a C. elegans worm with a barcode molecule. A RAB-3 promoter was used to control the expression of a mRNA barcode, which had a 3′-terminal MS2 stem loop region, in the neurons of C. elegans. The same neurons also expressed a synaptic protein attached to a MS2 dimer protein, which binds to the mRNA barcode MS2 stem loop and transported the barcode along with it to synaptic regions.

FISH imaging was used as shown in (C) to analyze the same region as shown in (B) of a transgenic worm that expressed both elements shown in (A). The mRNA barcode was detected using fluorescent FISH probes (magenta). The cell nuclei were visualized by DAPI staining (cyan).

The barcode was detected outside of the cell bodies and along the dendritic region of the nerves of the worm head and illustrate barcode transport.

### Example 7

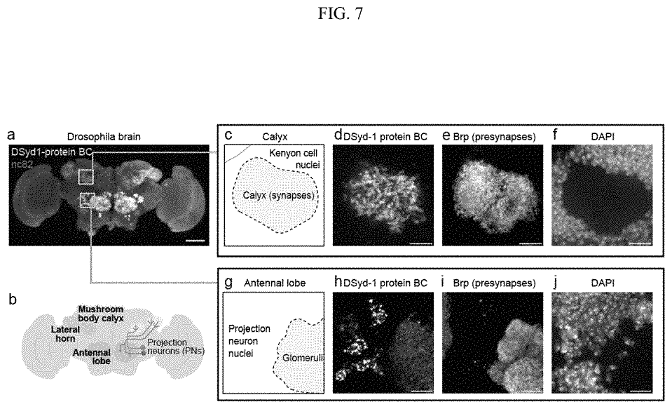

This example, as illustrated in FIG. 7, provides an exemplary process of mapping cellular components in neuronal cells. FIG. 7 provides an exemplary process of using transgenic barcode elements to label the synapses and cell bodies of neurons in the fruit fly Drosophila melanogaster.

Drosophila melanogaster transgenes were created using standard methods (injection by external provider Bestgene Inc, CA). A GH146 promoter was used to control expression of a barcode (abbreviated as BC) molecule linked to a synaptic protein DSyd-1 together with a scaffold molecule, in this exemplary embodiment a GFP (green fluorescent protein) molecule.

Brains of the flies were dissected and fixed in PFA. After PFA fixation, brains were permeabilized with Triton X-100 and incubated in a blocking buffer containing 5% BSA. A nc82 antibody against endogenous presynaptic protein was added to the brain for one day. The brains were washed and labeled with a secondary antibody conjugated to a fluorophore. The brains were washed again and labeled with oligo-conjugated antibodies against the presynaptic protein barcode and the postsynaptic protein barcode for one day. Brains were washed, labeled with DAPI and sequentially labelled with fluorophore-conjugated readout probes detecting the oligo-conjugated antibodies during the imaging experiments. Images were acquired in an anti-bleaching buffer using a confocal microscope.

(A-F) illustrates an exemplary embodiment of how a protein barcode was expressed and transported together with a presynaptic protein in antennal lobe projection neurons in adult drosophila brains. (A), illustrating the anatomical structures of the drosophila mushroom body calyx and (F), illustrating a schematic of mushroom body synapse structures and the expected locations for postsynaptic barcode molecules in relation to presynaptic boutons are provided as references.

Expression of the barcode-synaptic protein fusion was controlled with a cell-specific promoter for antennal lobe projection neurons. The barcode was detected using immunofluorescence against the recognition elements of the barcode, and is localized in the antennal lobe projection neuron cell bodies and synaptic structures in both the Mushroom body calyx and the lateral horn.

As shown in (C-F), antennal lobe projection neuron synapses were labeled with D-Syd1 protein barcode. (D) shows identifiable synaptic boutons corresponding to the calyx as illustrates in (C). Antibody labelling of the endogeneous presynaptic protein Brp in the same region was included as a control, and shows overlap with DSyd-1 protein barcode. DAPI staining of nuclei shown in (F) shows that DSyd-1 protein barcode is not found in the region corresponding to Kenyon cell nuclei.

(G-J) shows corresponding figures for the antennal lobe projection neurons. (H) shows DSyd-1 protein barcode expression both in projection neuron nuclei and less strongly stained in antennal lobe glomeruli. (J) shows antibody labelling of the endogeneous presynaptic protein Brp in the same region, which was included as a control and illustrates the location of antennal lobe glomeruli. DAPI staining of nuclei shown in (J) shows that DSyd-1 protein barcode is found in the region corresponding to antennal lobe projection neuron nuclei.

### Example 8

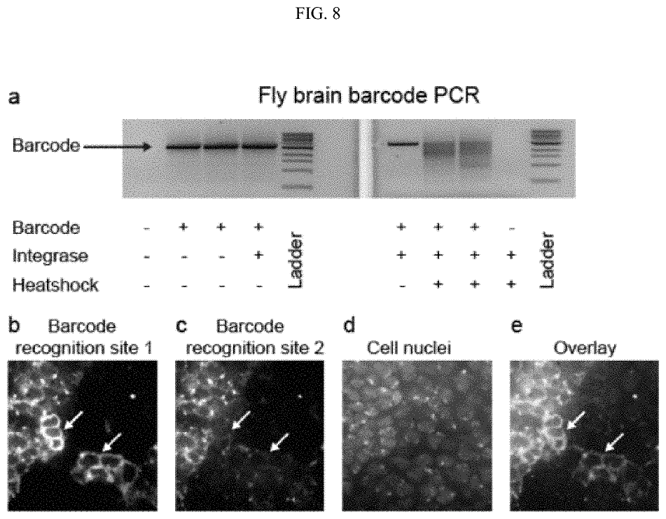

This example, as illustrated in FIG. 8, provides an exemplary process of mapping cellular components in Drosophila melanogaster.

Drosophila melanogaster transgenes were created using standard methods (injection by external provider Bestgene Inc, CA). Transgenic flies with a MB247-Gal4 driver for expression in mushroom body Kenyon cells were crossed with a transgenic UAS-Drep2-tag fly. Drep-2-tag consists of a postsynaptic protein fused to a protein tag carrying 6 different antibody epitope tags that can be recombined using the BxbI integrase. After crossing both transgenic flies, adult offspring with the correct genotype were collected.

Brains of the flies were dissected and fixed in PFA. After PFA fixation, brains were permeabilized with Triton X-100 and incubated in a blocking buffer containing 5% BSA. Antibodies against the postsynaptic tag and a nc82 antibody against an endogenous presynaptic protein were added to the brain for 2 days. The brains were washed and labeled with secondary antibodies conjugated to fluorophores. Brains were labeled with DAPI and imaged in an anti-bleaching buffer using a confocal microscope.

For recombination experiments, flies in fly vials were heatshocked for 1 hour at 37 C in a water bath. Afterwards, brains were dissected and stained with antibodies as explained above.

For PCR analysis of flies, heads of transgenic flies were digested in Proteinase K solution for 30 minutes at 65 C. PCR amplification and gel electrophoresis of the barcode insert were done using standard methods with primers at 5′ and 3′ invariable barcode regions.

An integrase was used to introduce a barcode into Drosophila melanogaster. To check the integration, as shown in (A) an agarose gel image of PCR-amplified barcode sequences that were extracted from flies expressing a barcode molecule and a Bxbl integrase was run. Integrase positive flies expressed Bxbl integrase under control of a heat shock promoter. Flies were heat shocked repeatedly during their larval stage, and DNA was extracted from adult flies. Controls are shown for flies without barcode or integrase, and with and without heat shock. A DNA ladder is included for size comparison. The blurred bands in barcode, integrase and heat shock positive flies, show editing of the otherwise sharp barcode band resulting in varying barcode lengths by deletion of barcode recognition sequences.

The transgenic files were imaged as shown in (B). The barcodes were expressed in a subset of neurons in the larval fly brain and edited following heat shock-dependent expression of Bxbl integrase. To detect the barcode two antibodies against different recognition sites of the barcode were used. Note that signal of recognition site 1 is present in some locations in the leftmost panel (B)-white arrows while signal of recognition site 2 is missing in (C)-white arrows. DAPI staining was included (second to right panel) to show locations of cell nuclei in the overlay of all channels (rightmost panel).

### Example 9

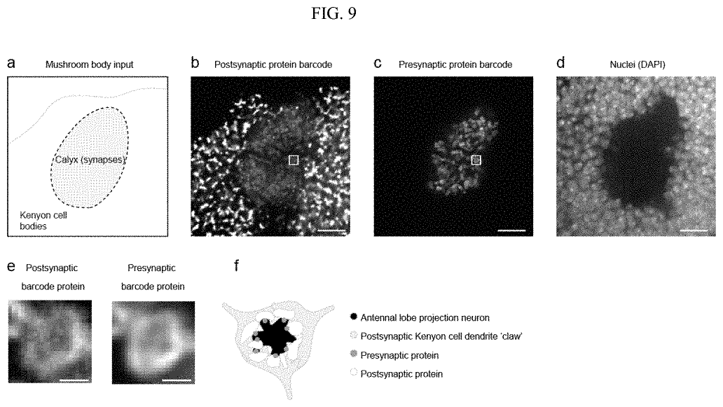

This example, as illustrated in FIG. 9, provides an exemplary process of mapping cellular components in neuronal cells. FIG. 9 provides an exemplary process of using transgenic barcode elements to label the synapses between different neurons in the fruit fly Drosophila melanogaster.

Transgenic flies with a GH146-Gal4 and a MB247-lexA driver for expression of two transgenes in antennal lobe projection neurons and mushroom body Kenyon as well as a Bxbl integrase under the control of a heatshock promoter cells were crossed with a transgenic UAS-D-Syd-1-barcodeA, lexAop-Drep2-barcodeB fly. DSyd-1-barcodeA consists of a presynaptic protein fused to a protein tag carrying 6 unique antibody epitope tags, which are not included in the postsynaptic protein barcode, that can be recombined using the BxbI integrase. Drep-2-barcodeB consists of a postsynaptic protein fused to a protein barcode carrying 6 unique antibody epitope tags, which are not included in the presynaptic protein barcode, that can be recombined using the BxbI integrase. After crossing both transgenic flies, adult offspring with the correct genotype containing all genetic elements were collected.

Brains of the flies were dissected and fixed in PFA. After PFA fixation, brains were permeabilized with Triton X-100 and incubated in a blocking buffer containing 5% BSA. Oligonucleutide-conjugated antibodies against presynaptic protein barcode and postsynaptic protein barcode were added to the brain for one day. Brains were washed, cleared for increased imaging quality, labeled with DAPI and sequentially labeled with fluorophore-conjugated readout probes detecting the oligo-conjugated antibodies during the imaging experiments. Images were acquired in an anti-bleaching buffer using a confocal microscope.

(A-F) illustrates an exemplary embodiment of how a protein barcode was expressed and transported together with presynaptic proteins in antennal lobe projection neurons and postsynaptic proteins in Kenyon cells in adult drosophila brains. (A), illustrates the anatomical structures of the drosophila mushroom body calyx and (F), illustrates a schematic of mushroom body synapse structures. The expected locations for postsynaptic barcode molecules in relation to presynaptic boutons are provided as references.

Expression of the barcode-synaptic protein fusion was controlled with a cell-specific promoter for antennal lobe projection neurons and Kenyon cells, respectively. The barcode was detected using immunofluorescence against the recognition elements of the barcode, and shows an overlap between presynaptic and postsynaptic protein barcodes in synaptic structures in the calyx.

As shown in (B), postsynaptic protein barcode expression was imaged in the calyx. Both cell bodies and synapses were labelled. (C) shows the same region with presynaptic protein barcodes labelled. Further, as shown in (D), the locations of cell nuclei in the same region labeled with the fluorescent dye DAPI.

(E) illustrates a magnified version the locations of protein barcodes are shown for an exemplary region synapse in the Mushroom body calyx. The magnified region corresponds to the white rectangle shown in (B-C). The same pre- and postsynaptic proteins as shown in (B-D) were localized in a characteristic pattern for synapses of projection neurons and Kenyon cells and show a spatial overlap between pre- and postsynaptic protein barcodes.

### Example 10

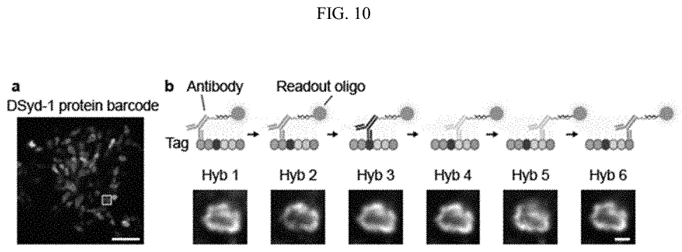

This example, as illustrated in FIG. 10, provides an exemplary process of using antibodies labeled with oligonucleotides to sequentially image individual barcode elements at the same synaptic region in a PFA-fixed Drosophila melanogaster brain. The antibodies labeled with oligonucleotides used had the epitope tags and oligonucleotide sequences according to Table 1.

TABLE 1

| | | SEQ |
|---|---|---|
| Epitope tag | Oligonucleotides | ID NO |
| EE | AAATTCCACACCTACCACAAA | 2 |
| ALFA | AAACCCTACTAACCATCTAAA | 3 |
| E | AAACCTCTCATCTTCCACAAA | 4 |
| DYKDDDDK (SEQ ID NO: 1) | AAACCTAAACATCTCCTCAAA | 5 |
| VSVG | AAAAATCCCTCACCACACAAA | 6 |
| OLLAS | AAATCCACCCATATCACTAAA | 7 |

(A) illustrates an exemplary embodiment where a GH146 promoter was used to control expression of a barcode molecule, which is linked to a synaptic protein DSyd-1 together with a scaffold GFP molecule. The image shows the presynaptic region of the GH146-positive antennal lobe projection neurons found in the mushroom body calyx.

(B) illustrates the sequential imaging individual barcode elements using antibody conjugated to unique oligonucleotide sequences. During the experiment, antibody-conjugated oligonucleotide sequences are bound by specific, fluorophore-labeled oligonucleotides sequences (‘readout oligos’). After removal of the fluorescent signal recorded in hybridization round 1, the next readout oligo is bound to another epitope tag, until all epitope tags are imaged. Exemplary images correspond to the white rectangle shown in (A) and show the signal for all 6 unique epitope tags imaged with 6 unique readout oligos at the same synaptic location.

### Example 11

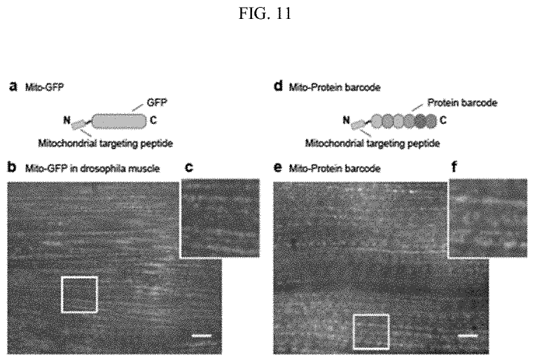

This example, as illustrated in FIG. 11, provides an exemplary process of labelling organelles with barcode molecule in a transgenic model organism.

(A) illustrates a mitochondria-targeting peptide attached to a GFP molecule. The targeting peptide is Cox 8, the mitochondrial presequence of human cytochrome c oxidase subunit VIII and had the sequence

(SEQ ID NO: 8)
MSVLTPLLLRGLTGSARRLPVPRAKIHSLPPEGKLIDYDVPDYASLMS.

(B) illustrates an exemplary image of a larval Drosophila melanogaster muscle labeled with a mitochondrially targeted GFP molecule.

(C) illustrates a magnification corresponding to the white rectangle in (B), showing fluorescently labeled mitochondria in the tissue.

(D) illustrates a mitochondria-targeting peptide attached to a barcode molecule that has, in this exemplary embodiment, 6 unique epitope tags.

(E) illustrates an exemplary image of a larval Drosophila melanogaster muscle labeled with a mitochondrially targeted protein barcode molecule, similar to the mitochondrially targeted GFP shown in (B-C).

(F) illustrates a magnification corresponding to the white rectangle in (E), showing fluorescently labeled mitochondria in the tissue.

### Example 12

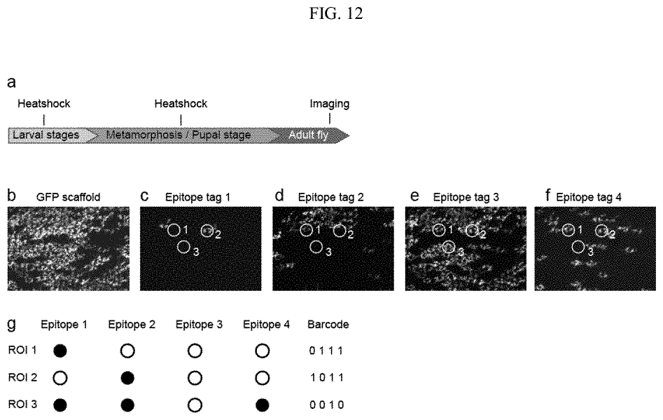

This example, as illustrated in FIG. 12, provides an exemplary process of mapping the diversity of spatially distributed neuronal barcodes.

(A) illustrates an exemplary timeline of an imaging experiment with transgenic Drosophila melanogaster, which express barcode molecules that are transported to synapses and a heat shock-dependent recombinase to create barcode diversity. In this example, flies are heat shocked during their larval and pupal stages. At the adult stage, fly brains are dissected, PFA-fixed, labeled with oligo-conjugated antibodies and imaged using fluorescence microscopy.

(B) illustrates exemplary confocal microscopy image of a GFP molecule used as a scaffold protein for the protein barcode in the presynaptic region of the L3 neurons in the optic lobe of the fly.

(C-F) illustrate exemplary confocal microscopy images of protein-barcode signals in the same presynaptic region of the L3 neurons in the optic lobe of the fly as shown in (B). Protein barcodes were imaged using fluorescently-labeled oligonucleotide probes specific for the oligonucleotides conjugated to the epitope antibodies. In the same imaging region, presynaptic terminals corresponding to individual neurons are detected in some imaging rounds, but not in others in line with the heatshock-induced recombination of the protein tags.

(G) illustrates an example of barcode information based on the images shown in (C-F). In regions of interest (ROIs) corresponding to numbered circles shown in (C-F), signal is detected in some imaging rounds and indicated with a white circle in the overview. ROIs that do not have signal in the imaging rounds shown in (C-F) are indicated as black circles. Based on the ROI information listed in the overview for epitope tags 1-4, a barcode is constructed, with “0” corresponding to no signal detected at the particular ROI where the epitope tag was deleted, and “1” corresponding to signal detected at the particular ROI where protein barcode molecules with the unedited epitope tag were present.

### REFERENCES

The following references are incorporated by their entirety.
Tyler J. Bechtel, Tamara Reyes-Robles, Olugbeminiyi O. Fadeyi, and Rob C. Oslund. Strategies for monitoring cell-cell interactions. Nature Chemical Biology volume 17, pages 641-652 (2021).
International PCT Patent Application No. PCT/US2014/036258, file Apr. 30, 2014, and titled MULTIPLEX LABELING OF MOLECULES BY SEQUENTIAL HYBRIDIZATION BARCODING.
U.S. patent application Ser. No. 15/713,597, filed on Sep. 22, 2017, and titled RECORDING AND MAPPING LINEAGE INFORMATION AND MOLECULAR EVENTS IN INDIVIDUAL CELLS.
Shah, S., Lubeck, E., Zhou, W., Cai, L., (2016). In Situ Transcription Profiling of Single Cells Reveals Spatial Organization of Cells in the Mouse Hippocampus. Neuron.
Hamming, Richard Wesley (1950) “Error detecting and error correcting codes.” Bell System Technical Journal. 29(2): 147-160.

1. A method for mapping spatial distribution of one or more cellular components comprising:
providing one or more cells;
providing one or more barcodes, wherein each barcode identifies a cell or cellular component in or near the one or more cells;
localizing each barcode to a site of a cellular component;
detecting each barcode and its location in the one or more cells; and recording one or more locations of each barcode to map the spatial distribution of the one or more cellular components.

2. The method of claim 1, wherein at least one cellular component is intracellularly or intercellularly distributed.

3. (canceled)

4. The method of claim 1, wherein at least one barcode comprises one or more barcode elements.

5. The method of claim 1, wherein at least one barcode comprises endogenous or exogenous elements in a cell.

6. The method of claim 5, wherein the one or more barcode elements comprise barcode recognition sequences that are recognized by one or more recognition molecules.

7. The method of claim 6, wherein the barcode recognition sequences are adjacent to or are flanked by one or more barcode elements capable of recruiting a molecule capable changing the barcode elements.

8. The method of claim 7, wherein the molecule capable of changing the barcode is selected from nucleases, recombinases, integrases, Cas9 nucleases, and combinations thereof.

9. The method of claim 1, wherein the recognition sequence comprises a nucleotide sequence having a length between about 10 bp to about 20,000 bp.

10.-11. (canceled)

12. The method of claim 1, wherein the recognition sequence comprises a nucleotide sequence, a protein coding nucleotide sequence, multiple repeats of the same protein coding nucleotide sequence, a codon optimized protein coding nucleotide sequence, a protein or any combination thereof.

13. The method of claim 1, wherein the recognition sequence comprises one or more repeated amino acid sequences.

14. The method of claim 13, wherein the repeated amino acid sequences comprise repeats of epitope tags.

15. (canceled)

16. The method of claim 1, wherein the recognition sequence comprises repeated, variable nucleotide sequences, coding for the same amino acid sequence.

17.-18. (canceled)

19. The method of claim 1, wherein each barcode is an exogenous set synthesized outside the cell.

20. The method of claim 1, wherein the barcodes comprise unique combinations of barcode elements.

21. (canceled)

22. The method of claim 1, wherein one or more barcode elements are error correction elements.

23. The method of claim 1, wherein the barcodes are expressed in the cell, wherein the barcodes are expressed as RNA and translated to protein.

24. (canceled)

25. The method of claim 1, wherein the barcodes are expressed under control of constitutively active promoter sequences, cell-specific promoter sequences, drug-inducible, light-inducible, or temperature-dependent promoter-sequences, or any combination thereof.

26. The method of claim 1, wherein the barcodes are introduced to two or more cells, wherein the two or more cells interact with each other in a tissue sample.

27. The method of claim 1, wherein the barcodes comprise one or more orthogonal barcode sets, wherein each barcode in the barcode set comprises one or more detection site that differs in binding from the one or more binding sites of a different barcode.

28.-32. (canceled)

33. The method of claim 1, wherein one or more barcodes are linked to one or more scaffold proteins or nucleic acid scaffolds capable of localizing the barcodes.

34.-36. (canceled)

37. The method of claim 1, wherein the method further comprises mapping intercellular and/or intracellular spatial distribution of one or more cellular components.

38. The method of claim 1, wherein the intercellular and/or intracellular one or more cellular components are spatially distributed between two or more cells in a population.

39.-40. (canceled)

41. The method of claim 1, wherein the recording and mapping of intercellular and/or intracellular spatial distribution of one or more cellular components of cells comprises relating the molecular properties of one or more transport molecules to one or more locations of the barcodes.

42.-43. (canceled)

44. The method of claim 1, wherein the recording and mapping intercellular spatial distributions of one or more cellular components comprises applying a set of probes to one or more cells.

45. The method of claim 1, wherein the probes comprise antibodies or peptides that can bind specific recognition sequences of the barcode.

46. The method of claim 1, wherein the probes are selected from DNA, RNA, small molecules that can bind specific recognition sequences of the barcode, and combinations thereof.

47. The method of claim 1, wherein the one or more probes bind to one or more target sites on one or more barcodes.

48.-55. (canceled)

56. The method of claim 1, wherein detecting the one or more barcodes comprises sequential rounds of imaging, sequencing, mass spectrometry, or any combination thereof.

57. The method of claim 1, wherein barcoded cellular components pass through a cell line to one or more progeny cells.


SEQUENCE LISTING (8 sequences)

SEQ ID NO: 1 - protein, 8 aa, synthetic construct
  REGION 1..8: note = Synthetic: Epitope
DYKDDDDK

SEQ ID NO: 2 - DNA, 21 bp, synthetic construct
  misc_feature 1..21: note = Synthetic: Epitope - EE
  source 1..21: mol_type = other DNA
aaattccacacctaccacaaa

SEQ ID NO: 3 - DNA, 21 bp, synthetic construct
  misc_feature 1..21: note = Synthetic: Epitope - ALFA
  source 1..21: mol_type = other DNA
aaaccctactaaccatctaaa

SEQ ID NO: 4 - DNA, 21 bp, synthetic construct
  misc_feature 1..21: note = Synthetic: Epitope - E
  source 1..21: mol_type = other DNA
aaacctctcatcttccacaaa

SEQ ID NO: 5 - DNA, 21 bp, synthetic construct
  misc_feature 1..21: note = Synthetic: Epitope - DYKDDDDK
  source 1..21: mol_type = other DNA
aaacctaaacatctcctcaaa

SEQ ID NO: 6 - DNA, 21 bp, synthetic construct
  misc_feature 1..21: note = Synthetic: Epitope - VSVG
  source 1..21: mol_type = other DNA
aaaaatccctcaccacacaaa

SEQ ID NO: 7 - DNA, 21 bp, synthetic construct
  misc_feature 1..21: note = Synthetic: Epitope - OLLAS
  source 1..21: mol_type = other DNA
aaatccacccatatcactaaa

SEQ ID NO: 8 - protein, 48 aa, synthetic construct
  REGION 1..48: note = Synthetic: Cox 8
MSVLTPLLLRGLTGSARRLPVPRAKIHSLPPEGKLIDYDVPDYASLMS